(19) United States
(12) Patent Application Publication
(10) Pub. No.: US 2018/0266956 A1
(43) Pub. Date: Sep. 20, 2018

(54) A METHOD FOR DETECTION AND SELECTION OF HYBRIDOMA CELLS PRODUCING THE DESIRED ANTIBODIES

(71) Applicant: WROCLAWSKIE CENTRUM BADAN EIT+ SP. Z O.O., WROCLAW (PL)

(72) Inventors: Tomasz LIPINSKI, WROCLAW (PL); Michal SKOWICKI, Walbrzych (PL); Artur BEDNARKIEWICZ, Wroclaw (PL)

(21) Appl. No.: 15/759,822

(22) PCT Filed: Sep. 13, 2016
(86) PCT No.: PCT/PL2016/050037

§ 371 Date: Mar. 13, 2018

(30) Foreign Application Priority Data
Sep. 15, 2015 (PL) P.413909

(51) Int. Cl. G01N 21/64; C07K 16/00

(52) U.S. Cl. CPC G01N 21/6428; C07K 16/00; C07K 2317/14; G01N 21/6458; G01N 2021/6441

(57) ABSTRACT

The object of the invention is the method of detection and selection of hybridoma cells capable to produce desired antibodies, comprising seeding the hybridoma cells in a culture vessel with biofunctionalized surface, containing culture medium, adding biofunctionalized luminescent labels and incubating such hybridoma cell culture, followed by optical detection of hybridoma cells producing desired antibodies by reaction of he biofunctionalized luminescent labels with the antibodies and detecting a luminescent label's signal border around hybridoma cells producing desired antibodies, and further separation in situ hybridoma cells producing the given antibody type from the rest of the cells.

The present invention relates to the method of detecting and selecting hybridoma cells which are capable to produce antibodies of desired specificity from among other cells in a culture. The selection process of hybridoma cells is a key step in the process of the production of monoclonal antibodies. Due to the versatile application of those antibodies in medicine, diagnostics, biotechnology, and other fields, methods which enable their production in afaster, cheaper and more efficient way are of high significance for medical diagnostics and therapeutic strategies' development.

Monoclonal antibodies play the main role in the modern targeted therapy. They belong to group of biological drugs, called biopharmaceuticals. Targeted molecular treatment consists in defining the appropriate molecular target and then selecting the appropriate medicine active towards the selected target, and selecting a group of patients that benefit from the treatment. Application of monoclonal antibodies in treatment directed at molecular targets brought significant benefits in improving treatment results of many serious diseases. Particularly, they found applications in cancer therapy and in transplantology.

The process of obtaining the monoclonal antibodies consists in breeding a monoclonal (derived from a single cell) hybridoma cell line capable to produce antibodies of identical amino acid sequence, thus of identical specificity. Hybridoma is formed by combining a B lymphocyte and a cell of mouse myeloma, and contains genetic material of both cells. The B lymphocyte determines the kind and the specificity of the produced antibodies, while the myeloma cell provides infinite cell multiplication (immortality) of the hybridoma cell.

The classic method of monoclonal antibodies production involves ainjecting a mouse with an antigen in order to activate its immunological system to produce B cells, reactive to the antigen used for immunization and capable to produce antibodies specific for that antigen. In the next step, cells from the mouse's spleen that contain, among others, the B cells specific for the antigen used for immunization are isolated and subjected to fusion with the myeloma cells. In the classical method, known from scientific literature, the initial selection of hybridomas takes place in a selective HAT medium, where the myeloma cells are not able to survive. Only hybridoma cells can survive and proliferate in the medium. Traditionally, mouse myelomas used for cell fusions contain a genetic defect preventing the synthesis of the enzyme necessary for their functioning. The gene that enables the synthesis of this enzyme is introduced to the hybridoma by the fusion with a lymphocyte B, and that's why only the cells formed in that process are able to survive. The B cells are not capable of prolonged growth in HAT medium, either. In the next step, the hybridoma cells are subjected to cloning process in order to obtain a cell line descending from a single cell and producing identical antibodies. Further on, the specificity of the produced antibodies is examined, which enables indication of the cell line sought for.

The cloning process is laborious and time-consuming, and according to the present state of the art, it is carried out by appropriate dilution of the cell suspension (usually after one or more days after fusion) and seeding them in in multiwell plates such as to get one hybridoma cell in each well statistically. The plates are placed in an incubator for a few days in order to let the hybridoma cells to proliferate. The plates are monitored under a microscope, and the growth stage and the number of colonies per well are determined. Presence of more than one colony in a well is interpreted as culture resulting from the number of cells equal to the number of colonies, i.e. not monoclonal. The specificity of the produced antibodies is determined by means of immunoenzymatic tests (ELISA) the analysis applies to the selected or all culture plate wells, where the number of cells is sufficient to produce the amount of antibodies that can be analysed with those methods. The cells from the wells, where the presence of the antibodies being sought for was confirmed, are subjected to further—sometimes multiple—cloning procedure. This is done to ensurethat the derived lines descend from the one cell. After the last cloning, the obtained lines are transferred to larger culture vessels and propagated to high cell density. Supernatants harvested from the above mentioned cultures are the source of monoclonal antibodies that are further purified with commonly known methods.

In another embodiment of the monoclonal antibodies production process, the hybridoma clone cells are inoculated intraperitoneally into appropriately prepared mice. This operation results in theimplementation and growth of hybridoma cells in the mouse's intraperitoneal cavity with concurrent production of large amounts of ascites fluid. The ascites fluid is the source of monoclonal antibodies whose concentration is much higher than in case of supernatants from cell cultures. Purification of antibodies from ascites is carried out similarly to that of cell culture supernatants, by known methods. Therefore, a number of key steps in the monoclonal antibodies production process can be indicated:
1) Preparation of the antigen for immunization,
2) Immunization of animals (usually carried out by a number of injections at intervals of a few weeks),
3) Isolation of splenocytes from the spleens of immunized animals, and then fusion with myeloma cells - obtaining the hybridomas,
4) Hybridomas cloning and selection (usually carried out at least twice),
5) Proliferation of clones, production and purification of antibodies.

An essential drawback in the currently applied cloning and selection procedure is the fact, that the excretion of antibodies can be examined only after a certain time from cell seeding, when the colony has grown enough to produce the appropriate amount of antibodies that can be analysed with ELISA method. The procedure is time- and labour-consuming, and requires much engagement from the employees carrying out cloning and selection. One of the problems is the necessity of multiple ELISA analyses, which results from the uneven (i.e. not synchronized) growth of hybridoma colonies, and is associated with the need for the manual identification of wells for analysis (1 plate comprises typically 96 wells) and tiresome pipetting. It is possible to perform multiple ELISA analyses of the whole plate, which simplifies pipetting significantly (multichannel pipettes), but this is associated with the increased consumption of materials and reagentsDespite the existence of many technologies that enable labelling and sorting cells based on that labelling, none of them meets untypical requirements and challenges posed by hybridoma cells. For example, the cells can be labelled with organic luminophores and then sorted in a cell sorting apparatus that directs cells to different containers, based on the fluorescent signature coming from the labels attached to the cell. This technique allows to isolate and separate individual cells from a large population of cells in a very efficient way. However, in the case of hybridomas, the labeling must concern the antibodies produced by them, and not the cell structural elements themselves. These antibodies, however, are not permanently bound/anchored to the hybridoma cell membrane and are released into the environment. Only minor population of hybridomas, apart from the secreted antibodies present also antibodies permanently anchored to the membrane, and can be selected with a sorter on that basis.

The efficient application of a sorter can be achieved using specially prepared, genetically modified myeloma cells that provide constitutive expression of antibodies on the cell membrane surface of the obtained hybridomas, as disclosed in U.S. Pat. No. 7,148,040. The disadvantage of using the sorter is the increased number of manipulations that the cells are subjected to (the necessity of their pipetting, labeling, washing, etc.). A sorter is also a relatively expensive piece of equipment, and due to its complex operation requires highly qualified personnel. Furthermore, additional manipulations increase the risk of infecting the culture with bacteria.

The described above classical method of obtaining monoclonal antibodies is a very lengthy and costly process since it is carried out completely manually without the possibility of automation. Particularly, this apply to the selection of hybridomas producing the antibodies of defined specificity.

Unlike the methods of direct cell labelling (i.e. Molecules permanently bound with the cell structure), the selection of appropriate hybridomas requires labelling the molecules released by the cells into the environment. One of few methods that may be used for that purpose is the technique known as ELISPOT. It is a method used in scientific research in immunology, oncology, biotechnology, in medicine, laboratory diagnostics, etc. It is used, among others, to examine the cellular response by the possibility to detect a single cell producing the analysed protein substance (e.g. cytokines, chemokines, receptor proteins, antibodies). It consists in culturing cells in a culture vessel whose surface is covered with capturing antibodies. The substances excreted by active cells are bound by the capturing antibodies in the direct vicinity of those cells. Then the cells are removed and the bound substances detected in a way like in classic ELISA method, i.e. by reacting with appropriate antibodies specific for the examined substances and with reporter antibodies (e.g. conjugated with enzymes or fluorophores). An appropriate device reads and analyses the number of coloured spots, where each spot is representing a trace after a single cell excreting the substance being the object of analysis. The ELISPOT method, however, is not suitable for the selection of positive hybridomas. It is mainly because cells are irreversibly lost during the analysis process, that prevents establishment of a cell line. which actually prevents developing any

[symbol]

U.S. Pat. No. 7,622,274 discloses a method to purify one or more cells in a cell population (e.g. producing humanized antibodies) based on their excreted products (e.g. antibodies). The method according to the patent referred to, comprises (a) immobilizing the cells on a capture matrix that is capable of binding the product excreted by the desired cells with the marker that binds selectively with the excreted product and emits signal in the form of light - indicating at the same time (e.g. by its characteristic fluorescence) the cells sought after, (b) illuminating the cell population, (c) detecting at least one property of the emitted light out of which at least one identifies the products situated on the capture matrix, (d) determining the value of at least one property of the emitted light in order to determine the excretion profile of one or more selected cells in the whole population. In the presented method, a lethal dose of light radiation is used in order to isolate one or more selected cells. The method according to the invention was used to select and purify hybridomas. In the first step, a biotinylated antigen was added to the hybridomas producing the desired antibody (IgG), followed by streptavidin with fluorescent marker AlexaFluor-532 attached, furthermore all cells were non-specifically labelled with Styo13, and then the cells were washed and irradiated with electromagnetic radiation of 485 nm and 532 nm wavelengths in order to photo-excite Styo13 and AlexaFluor-532 labels. The fluorescence from the system was detected by means of a CCD camera equipped with optical filters, cutting off radiation of the wavelength smaller than 530 nm and 645 nm, respectively. The bi-colour image resulting from the irradiation was analysed manually, and only those cells that exhibited red fluorescence near them (AlexaFluor-532) were selected. The areas where no red fluorescence was found were subjected to irradiation with a high (destructive) dose of laser radiation at 532 nm wavelength from a semiconductor pulsed laser. The radiation dose caused photomechanical ablation and was lethal for the cells. This way, a purified product in the form of selected hybridomas capable of producing the desired antibody was obtained. In the method referred to above, the application of the same wavelength to select the appropriate cells and to destroy incompliant cells can cause some difficulties. During identification of hybridomas, the 532 nm radiation can lead to destruction thereof when the light dose is exceeded. Furthermore, the applied organic fluorescent dyes, due to their character and due to the absorbed spectrum range, are characterised with the occurrence of a photobleaching effect, demonstrate weak luminescence stability in time, and short lifetime of the emitted electromagnetic radiation. Furthermore, irradiation with 532 nm wavelength during selection process, can induce the cell auto-fluorescence effects, that may interfere with the readout or reduce the optical contrast necessary to differentiate positive hybridomas from other cells. The wavelength of 532 nm used to detect signal from positive hybridomas can also induce auto-fluorescence of the culture plate or proteins present in the medium. In consequence, the number of false positive cells grows that translates into the impaired selection effectiveness and the complexity (necessity to repeat the purification) of hybridomas selection process. Using laser radiation of high power density requires raster scanning of the culture, which makes the selection process longer and requires application of galvano mirrors. All these factors adversely influence the effectiveness of desired cells selection at the cost of the whole process. Furthermore, a significant disadvantage of the solution presented in patent U.S. Pat. No. 7,622,274 referred to herein is the necessity of intensive,repetitive, sometimes even 10 times, washing of the culture vials in order to remove the labels from the culture medium. Washing is necessary since the fluorescent labels which are not bound with the antibodies excreted by the hybridoma cells and remain in the culture medium, generate high signal and thus prevent identification of the positive hybridomas. The necessity of washing is connected with the increased costs (medium consumption), higher amount of labour, and risk of infecting the culture. The most important disadvantage is, however, the fact that the hybridomas are not adherent cells and therefore, washing the culture causes the risk of their detachment and loss.

Patent application US20080009051, in turn, discloses another method of selection of desired cells from a mixture of many different cells. The method is implemented by immobilizing the selected cells and washing the other away. The process is carried out in the following steps: placing a cell mixture in a light-sensitive medium, selecting at least one cell for immobilization, irradiating the light-sensitive medium near the selected cells with appropriate radiation in order to change the condition of the light-sensitive medium in order to immobilize the selected cells. The cell selection process comprises optical observation of cells, selection of the desired cells and localized modification of the culture vessel surface in order to immobilize the said cells. The observation may be based on detecting the properties of light from fluorescent labels attached to the desired cells in order to identify them. From among the fluorescent labels referred to, all are based on visible spectrum absorption and emission, with small spectral intervals. Immobilization of selected cells takes place by irradiating the light-sensitive material included in the culture medium with UV radiation of 375 nm wavelength. It is the most important disadvantage of this solution since the UV band light can negatively affect or kill cells by damaging DNA, proteins or enzymes. Besides, the fluorescent markers operating in the visible spectrum and used for detection, are characterized by the occurrence of photobleaching/fading effect, weak luminescence stability and short lifetime of the emitted electromagnetic radiation. Furthermore, the method referred to does not offer the possibility to detect hybridoma cells producing desired antibodies since it claims application of absorbing or fluorescent markers that mark the cells and not the products excreted to the environment, like in case of hybridoma cells producing specific antibodies.

American patent application No. US2007243573 discloses a method to immobilize cells by increasing their adhesion to the surface of the culture vessel in the result of irradiation with ultraviolet light in 330-410 nm wavelength range. In the said patent application, a device was used to generate light patters on a surface of tissue culture vial , by a system of micro-mirrors in such a way that the light reflected by the projector “freezes” the positive/desired cells (positive process). Using ultraviolet radiation for that purpose can adversely influence the detected cells, destroying the DNA and causing damage thereof. What's more, the sensitivity of the applied detection technique becomes limited due to the background luminescence induced with UV radiation, which significantly reduces contrast during observation. Furthermore, the solution to the problem of in-situ detection of cells producing desired antibodies is missing.

The technical problem faced by the present invention is to provide such a method of in-situ detection and selection of cells producing a desired antibody that is fast and effective (i.e. enables examination of a large population of cells in a short time), can be automated, does not expose the cells to the risk of removal by washing or damage by harmful electromagnetic radiation, and also provides a more sensitive detection of the sought cells due to a more favourable contrast between the background and the measured signal, and uses well developed, cheap technological solutions known from optical fibre telecommunications systems and optoelectronics. It is also desired that the method include labeling the cells (producing desired antibodies) in such a way that the labeling does not require many complex operations and can be carried out continuously for a longer period (e.g. not requiring the replacement of the medium or additional steps for colour development). Furthermore, it is desired that the method enables concurrent recognition of a few types of cells (e.g. producing different monoclonal antibodies sought for), and it should also enable discrimination of classes/subclasses of produced antibodies and estimation of productivity of the observed cells. Unexpectedly, the said technical problems have been solved by the present invention.

The object of the invention is the method to detect and select hybridoma cells producing desired antibodies, characterized in that it comprises the following steps:
a) hybridoma cells producing antibodies are placed in a culture vessel with a biofunctionalized surface, containing the culture medium,
b) biofunctionalised luminescent markers are added to the culture medium, and so obtained culture is incubated,
c) hybridoma cells producing desired antibodies are detected optically by the biofunctionalized luminescent marker's reaction with the antibodies,
d) hybridoma cells producing desired antibodies are separated in situ from other cells,
   wherein in step c) the luminescence of the luminescent labels, creating a shiny border (“halo”) around the hybridoma cells producing desired antibodies is detected. In a preferred embodiment of the present invention, step c) is repeated for the whole surface area of the culture vessel by means of raster scanning. Step c) of the method according to the present invention can be carried out in any way known from the art that enables systematic examination of the given surface in a one-off and sequential way, i.e. from one area to another. Alternatively, it is also possible to carry out the scanning “on the fly”, where, by definition, each fragment of the culture is irradiated unless a luminescent signal coming from nanoluminophores is earlier detected. Then, both scanning in search of luminescence indicating finding the hybridoma, and the irradiation with UV in order to initialize the photo-destruction process, might take place simultaneously in time, but separately in space. Alternatively, in case of selecting suitably small culture well, suitably small magnification of the optical system, and a photodetection system of suitable sensitivity and resolution, the irradiation of the specified area of the well could take place in one step. In another preferable embodiment of the present invention, the cells are incubated with a photosensitizer or a photosensitizer precursor prior to step c). In another preferable embodiment of the present invention, step d) is carried out by means of photodynamic reaction controlled in space. The step of separating hybridoma cells producing desired antibodies from the remaining cells can be, alternatively, implemented by any method known in the art, such as photo-activated positive process, photo-activated negative process or using a micromanipulator or high-resolution laser ablation. In the preferred embodiment of the present invention, luminescent markers demonstrate absorption and/or emission in the spectral range not overlapping with photosensitizer photoexcitation and/or absorption bands. Nanoluminophores, semiconductor nanocrystals (Ag₂S, Ag₂Se, PbS, etc.), doped dielectric nanocrystals, fluorescent polymer spheres, or metal nanocrystals (AuNPs, AgNPs) can be used as luminescent labels. In another preferred embodiment of the present invention, luminescent labels comprise nanoluminophores demonstrating Stokes and/or anti-Stokes emission, doped with ions selected from the group comprising: Nd³⁺, Yb³⁺, Tm³⁺, Tb³⁺, Er³⁺, Eu³⁺, Ho³⁺, Pr³⁺, Dy³⁺, Sm³⁺, Yb³⁺-Tm³⁺, Yb³⁺-Tb³⁺, Yb³⁺-Er³⁺, Yb³⁺-Ho³⁺, Yb³⁺-Pr³⁺, Yb³⁺-Eu³⁺, Yb³⁺-Dy³⁺, Yb³⁺-Sm³⁺. In another preferred embodiment of the present invention, the surface of luminescent nanoluminophores is covered with one or more shells made of identical undoped material or indentical material doped with ions or combination of ions other than in the core of that marker. Preferably, the biofunctionalization of the culture vessel surface consisted in coating with an antibody recognizing antibodies produced by the hybridomas, and the functionalisation of the labels consisted in attaching the antigen used for immunization. Preferably, both surfaces, i.e. that of the culture vessel and that of the marker, were coated with the antigen. Alternatively, other types of biofunctionalization known in the art can be used, e.g. those selected from the group comprising the following variations:
1) an antigen attached to the surface of the medium of the culture vessel, a protein binding the given antibodies in a way not interfering with their paratopes, attached to the surface of the luminescent marker,
2) an antigen attached to the surface of the culture vessel, an antibody recognizing the given antibody, attached to the surface of the luminescent marker,
3) a protein binding the given antibody in a way not interfering with its paratope, attached to the surface of the culture vessel, an antigen attached to the surface of the luminescent label.

The proposed solution allows to automate the key steps of separating hybridomal cells producing the desired antibodies from the other cells in order to further proliferate the former, thus allowing to derive the hybridoma cell line producing the desired monoclonal antibodies in a fast and simple way—i.e. results in significant reduction in time and personnel's effort on selecting the hybridomas, reduces the cost of obtaining monoclonal antibodies, and improves the process of their production. The significant advantage of the technology described above is the possibility to define which cells should remain, and which should be removed in situ, without the need to breed all cells, including unproductive ones, and arduous analysis of the obtained clones in order to find clones producing the desired antibodies. Unlike in some other solutions, e.g. increased cell adhesion modulated with light the interaction of the UV light beam with positive cells is also eliminated. Photodynamic reaction technique used for thekilling cells which do not produce desired antibodies is easy to implement with generally available technical means, and allows for concurrent irradiation of many cells, thus parallel removal of large numbers of unnecessary cells, which further allows to reduce the time necessary to carry out the process on the whole plate. Photochemical reagents suitable for that purpose are cheap and are not directly (without intentional irradiation) harmful to the cells. Furthermore, the selection process can be initialized and completed in one culture vessel, which has a direct effect in reduced consumption of materials, work, and the risk of infecting the culture. Application of IR (particularly NIR) light for the identification and selection of cells does not have any adverse influence upon the cells, the surface of the vessel or the photosensitizers used. Furthermore, it enables application of equipment known from the well developed and very cheap fiber-optic communications technology, and technology of imaging with CCD/CMOS cameras. The application of up-converting nanoluminophores for detecting proper hybridomal cells does not negatively interfere with the process of photodynamic removal of unwanted hybridoma cells. It results from the fact that the radiation used for testing whether the cell is positive or negative (both excitation light and the light emitted by the label) does not overlap with the photosensitizer absorption range. The fluorescent labels used, display longer luminescence lifetimes, do not demonstrate photo-bleaching (loss of luminescence intensity over observation time), are not subject to photodegradation, demonstrate stable and relatively efficient luminescence which results in lack of parasitic auto-fluorescence on excitation with IR light. Furthermore, the passivation of the surface of the fluorescent labels allows to reduce unfavourable phenomena at the surface, which results in a weaker emission from the luminescent labels. These features allow for the detection of very weak signals due to lack of background signal (i.e. high signal to noise ratio is achieved). It means the possibility to detect proper hybridoma cells after a short time from seeding, when the quantity of produced antibodies is still too low for detecting them with standard techniques. Additionally, the disclosed technique allows detecting luminescent labels of different colours (a marker with one colour, attached with one AgX antigen will detect AbX antibodies, while a marker of a different colour, with AgY antigen, will detect AbY antibody, etc.). In this way, a single test will allow to detect two or more hybridoma cell types in the same culture at the same time. Furthermore, based on the size of the emerging fluorescent border (“halo”) around the labeled hybridoma cell and the dynamic of its formation it is possible to quantitatively determine the productivity of a single cell individually. Since the application of up-converting markers practically doesn't produce background signal, the productivity measurement by measuring the luminescence intensity can be a quantitative measurement (e.g. μg/ml), and not only a qualitative one. The proposed technique allows miniaturization and full automation of detection and selection at the cost of instruments significantly below the cost of cell sorters that, due to the lack of possibility to label hybridoma cells only, are not suitable for sorting typical hybridoma cell lines.

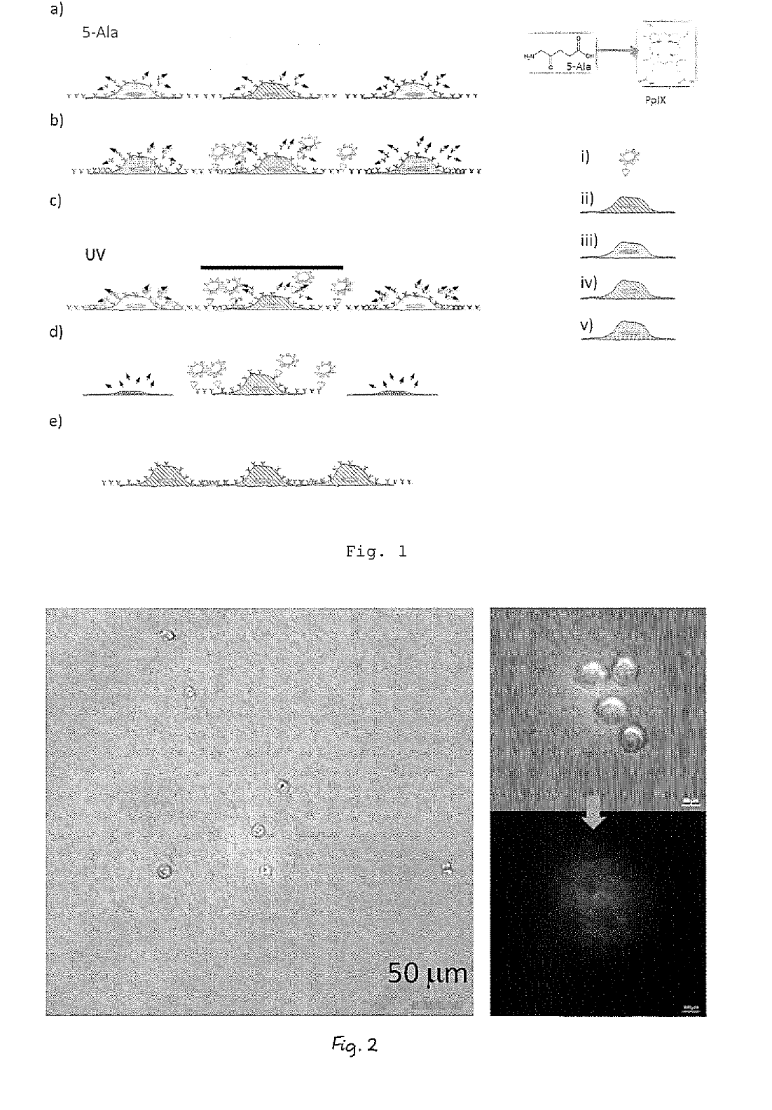

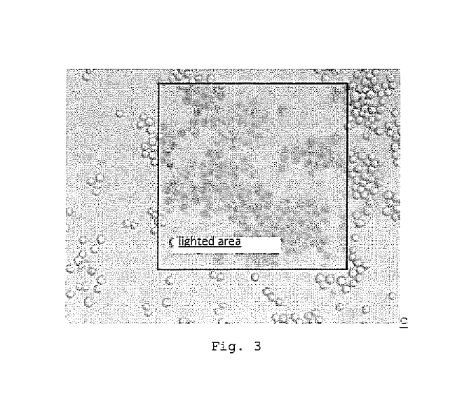

Exemplary embodiments of the present invention have been presented in the drawing, where FIG. 1 is a schematic representation of the method to detect and select hybridoma cells producing desired antibodies, FIG. 2 is a microscope photography illustrating excitation of luminescent label in the form of UCNPs, and FIG. 3 shows a photograph of hybridoma cells subjected to the effect of photosensitisation and UV light. In FIG. 1, symbols i) to v) indicate, respectively: i) luminescent or contrasting label, ii) positive hybridoma cell, iii) negative hybridoma cell, iv) positive hybridoma cell sensitive to destruction by UV light, v) negative hybridoma cell sensitive to destruction by UV light.

### EXAMPLE 1

Lableing hybridoma cells with biofuncitonalized up-converter nanocrystallites.

In the presented embodiment, hybridoma cells producing monoclonal antibodies (IgG₃K) recognizing bacterial lipopolysaccharide (LPS) from H.alvei 1186 were used. The cells were obtained as the result of fusion of SP2/0 cells with splenocytes obtained from Balb/c mouse immunized with killed H.alvei bacteria. The way of culture vessel preparation consisted in coating the surface of a 96-well plate (Maxisorp, Nunc) with antibodies recognizing mouse immunoglobulins (DAKO) in 0.1M carbonate buffer at pH 9.6 and concentration of 50 μg/ml by adding 100 μl of the solution to each well and incubating for 24 hours at 4° C. On so prepared plates, hybridoma cells were seeded at the density 5 cells/well. After hours, preparation of UNCPs covered with the antigen was added to the medium. The method of preparation of UNCPs (βNaYF₄: 0,5% Tm 20% Yb@ βNaYF₄) covered with the antigen consisted in the ligand (oleic acid) removal by means of 0.1M HCl followed by incubation in DMSO solution containing LPS H.alvei 1186 in acidic form (4 mg) previously pre-incubated with sodium dodecyl sulphate (1 mg) in DMSO. Such modified particles were then purified by multiple centrifugation, and eventually suspended in 0.9% NaCl with 1% BSA. After 72 hours after adding the nanoparticles to the culture, the effect of luminescent labeling of antibodies produced by positive hybridoma cells was observed, while negative cells did not demonstrate a luminescent border.

The further steps of the process consisted in detecting the “halo” (i.e. the luminescent border of antibodies marked with the labels, diagram in Fig. lb and the photograph in FIG. 2) in up-conversion mode (excitation in the near infrared band, detection of luminescence in the visible range of 470 nm—emission of Tm³⁺ ions) by means of fluorescent microscope (diagram in FIG. 1b and the photograph in FIG. 2); incubation of cells with δ-ALA acid and obtaining their photosensitization (cells producing protoporphyrin IX—Fig. 1a), and then selective irradiation in the cell's growth plane by means of a 375 nm projector (FIG. 1c).

In order to implement the step of selection of the labeled cells, a non-toxic photosensitizer precursor was added to the culture, which was accumulated by all cells. In the presented embodiment, photosensitizer precursor—delta-aminolevulinic acid in the concentration of 0.5-4 mM was added. Hybridoma cells have the intrinsic ability to convert that substrate (photosensitizer precursor) to protoporphyrin IX (photosensitizer) (FIG. 1a). The processes of adding the photosensitizer (precursor) (FIG. 1a) and luminescent and/or contrasting markers to label the antibodies (FIG. 1b) can be performed in any sequence without impact upon the obtained results.

The cells detection stage with the use of UCNPs required the application of light with the wavelength matching the nanoluminophores absorption in the red and near infrared range that does not overlap with the protoporphyrin IX absorption range. It did not interfere with the PDT process that required using UV/Vis photoexcitation. The application of luminophores allowed recognizing cells producing desired antibodies (FIG. 1) without the necessity to wash the unbound label away in order to improve the contrast between the background and the marked area around positive cells. FIG. 2 presents microscope photographs illustrating excitation of the label in the form of up-converting nanoparticles by irradiation with electromagnetic radiation of matching wavelength. Cells that were recognized as the ones producing desired antibodies were not irradiated with light matching the photosensitizer's absorption band, thus they were not destroyed but left for further proliferation. All other unwanted cells (i.e. those that did not produce desired monoclonal antibodies) were subject to irradiation with light at wavelengths matching photosensitizer used (FIG. 1c). As the result of such irradiation, photodynamic reaction was initiated, leading to localized release of free radicals and/or singlet oxygen, which resulted, in consequence, in destruction of unwanted cells (FIG. 1d).

FIG. 3 presents a representative microscope photograph (lens 20×, square with the side of 383 μm) of a concentrated hybridoma cell culture subjected to the effects of δ-ALA acid (2 mM) and UV light (385 nm, 5 J/cm², 510 mW/cm², irradiation for 10 s). The photograph was taken 30 minutes after the irradiation. Dead cells (in the centre of the square) were stained with TrypanBlue dye. After the irradiation, the vessel was moved to the incubator in conditions minimizing any accidental irradiation of the sensitized cells. The cells were left to proliferate without the need to remove dead cells at this stage. After ca. 3 days the cells sensitivity to light ceased, and the growth of cells could be monitored in white light microscope after that time. The process of detecting/selecting positive cells and destroying unwanted cells could be repeated if necessary, when the cells were still photosensitive (FIG. 1d and FIG. 1e).

Since negative hybridoma cells were killed, a single hybridoma cell producing desired antibodies remained in the well of the culture vessel. After its proliferation, a monoclonal hybridoma line producing desired antibodies was obtained (FIG. 1e). At this stage it was possible to wash the whole medium with the remains of dead cells delicately. This step completed the process of hybridoma selection.

### EXAMPLE 2

Example 2 was implemented in compliance with example 1, however, 2% Er 20% Yb containing βNaYF₄ nanocrystalline core and passive coat of βNaYF₄ were used as luminescent markers. The obtained halo was then green in color.

### EXAMPLE 3

Example 3 was implemented in compliance with example 1, however, 2% Nd³ doped βNaYF₄ core nanocrystals were used as luminescent labels. The obtained halo manifested marker emission in the near infrared band of ca. 870-900 nm under radiation from near infrared band (808 nm).

### Example 4

Example 4 was implemented in compliance with example 1, however, 2% Er 20% Yb doped βNaYF₄ core and 2% Nd³⁺ 20% Yb³⁺ doped βNaYF₄ shell: nanocrystals were used as luminescent labels. The obtained halo exhibited green label's emission (540 nm band) and near infrared band of ca. 870-900 nm under radiation from near infrared band (808 nm). The obtained halo exhibited green emission (540 nm band) also under radiation from near infrared band (980 nm).

### Example 5

Example 5 was implemented in compliance with example 1, however, both the luminophore particles and the culture vessel surface were coated with antigen molecules (LPS with H.alvei 1186). Plate surface coating was carried out in the following way: an LPS solution with H.alvei 1186 was prepared in a carbonate buffer (0.2 M, pH 9.6) with the concentration of 5 μg/ml, obtaining thorough dispersion of LPS in the buffer by means of ultrasounds, and then the solution was sterilized by filtration through 0.22 μm filter, the wells were filled with 100 μl of LPS solution and the plate was incubated overnight at +4° C. The plate was then washed with 0.9% NaCl solution, and the surface blocked by means of the culture medium supplemented with 10% bovine serum.

1. Method to detect and select hybridoma cells capable to produce desired antibodies, characterized in that it comprises the following steps:
a) hybridoma cells producing antibodies are placed in a culture vessel with a biofunctionalized surface, containing the culture medium,
b) biofunctionalized luminescent labels are added to the culture medium, and so obtained culture is incubated,
c) hybridoma cells producing desired antibodies are detected optically by the biofunctionalized luminescent label's reaction with the antibodies,
d) hybridoma cells producing desired antibodies are separated in situ from other cells,
wherein in step c) luminescent labels, creating a luminestent border around the hybridoma cells producing desired antibodies are detected.

2. Method according to claim 1, characterised in that step c) is repeated for the whole surface area of the vessel by means of raster scanning.

3. Method according to claim 1 or 2, characterised in that before step c), the cells are incubated with a photosensitizer or a photosensitizer's precursor.

4. Method according to claim 3, characterised in that step d) is carried out by means of photodynamic reaction controlled in space.

5. Method according to claim 3 or 4, characterised in that the luminescent labels demonstrate absorption and/or emission in the spectral range not overlapping with photosensitizer's photo-excitation and/or absorption bands.

6. Method according to any one of claims 1 to 5, characterised in that the luminescent markers comprise nanoluminophores demonstrating Stokes and/or anti-Stokes emission, doped with ions selected from the group comprising: Nd³⁺, Yb³⁺, Tm³⁺, Tb³⁺, Er³⁺, Eu³⁺, Ho³⁺, Pr³⁺, Dy³⁺, Sm³⁺, Yb³⁺-Tm³⁺, Yb³⁺-Tb³⁺, Yb³⁺-Er³⁺, Yb³⁺-Ho³⁺, Yb³⁺-Pr³⁺, Yb³⁺-Eu³⁺, Yb³⁺-Dy³⁺, Yb³⁺-Sm³⁺.

7. Method according to any one of claims 1 to 6, characterised in that the surface of luminescent labels is covered with one or more shells made of identical undoped material or identical material doped with ions or combination of ions other than in the core of that label.

8. Method according to any one of claims of I to 7, characterised in that the biofunctionalization of the luminescent label and/or the surface of the culture vessel comprised the formula:
i. the antibody recognizing the given antibody attached to the surface of the culture vessel, the antigen attached to the luminescent label surface, or
ii. the antigen attached to the surface of the culture vessel, the antigen attached to the luminescent label surface, or
